(19) United States
(12) Patent Application Publication
(10) Pub. No.: US 2010/0047313 A1
(43) Pub. Date: Feb. 25, 2010

(54) MEDICAL DEVICES HAVING A COATING FOR ELECTROMAGNETICALLY-CONTROLLED RELEASE OF THERAPEUTIC AGENTS

(75) Inventors: Tom PRIEBE, Shakopee, MN (US); Umang ANAND, Plymouth, MN (US); Lance GIBSON, Plymouth, MN (US); Les CLAPP, Mounds View, MN (US); Michael KUEHLING, Munich (DE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/544,577

(22) Filed: Aug. 20, 2009

Related U.S. Application Data
(60) Provisional application No. 61/091,115, filed on Aug. 22, 2008.

(51) Int. Cl. A61F 2/04; A61K 31/337; A61P 7/00

(52) U.S. Cl. 424/423; 514/449

(57) ABSTRACT

Medical devices having a coating comprising an ionic polymer for electromagnetically-controlled release of a therapeutic agent. Release of the therapeutic agent from the coating is facilitated or modulated by the application of an electromagnetic field to the medical device. Exposure to the electromagnetic field may cause the release of the therapeutic agent in various ways, including electrochemical changes in the ionic polymer, structural changes in the coating and/or ionic polymers, changes in the permeability of the coating, changes in the orientation of the ionic polymers, or motion of the ionic polymers. Also disclosed are methods for delivering a therapeutic agent using electromagnetic fields.

### TECHNICAL FIELD

The present invention relates to medical devices having a coating for the controlled-release of a therapeutic agent.

### BACKGROUND

Many implantable medical devices are coated with drugs that are eluted from the medical device upon implantation. For example, some vascular stents are coated with a drug which is eluted from the stent for treatment of the vessel and/or to prevent some of the unwanted effects and complications of implanting the stent. In such drug-eluting medical devices, various methods have been proposed to provide a mechanism for drug elution. However, there is a continuing desire for improved devices and methods for providing drug elution from medical devices.

### SUMMARY

In one aspect, the present invention provides a medical device comprising: (i) a coating comprising an ionic polymer; and (ii) a therapeutic agent retained by the coating; wherein the therapeutic agent is released from the medical device when the medical device is exposed to an electromagnetic field.

In another aspect, the present invention provides a method for delivering a therapeutic agent, comprising: (i) providing a medical device comprising: (a) a coating comprising an ionic polymer; and (b) a therapeutic agent retained by the coating; (ii) positioning the medical device at a site in a patient's body; and (iii) applying an electromagnetic field to the medical device, wherein the application of the electromagnetic field causes the release of the therapeutic agent from the medical device.

### BRIEF DESCRIPTION OF THE DRAWINGS

FIGS. 1A-1C show strut portions of a stent according to an embodiment of the present invention. FIG. 1A shows a cross-sectional side view of the strut portion. FIG. 1B shows the strut portion with therapeutic agents loaded into the coating. FIG. 1C shows the strut portion after implantation and exposure to an electromagnetic field.

FIGS. 2A-2C show strut portions of a stent according to another embodiment. FIG. 2A shows a top view of the strut portion. FIG. 2B shows a cross-sectional side view of the strut portion. FIG. 2C shows the strut portion after implantation and exposure to an electromagnetic field.

FIGS. 3A-3C show strut portions of a stent according to another embodiment. FIG. 3A shows a cross-sectional side view of the strut portion. FIG. 3B shows the strut portion after implantation and exposure to an electromagnetic field. FIG. 3C shows the strut portion with the lamellae sheets being broken apart by the swelling of the coating.

FIGS. 4A and 4B show strut portions of a stent according to another embodiment. FIG. 4A shows a cross-sectional side view of the strut portion. FIG. 4B shows the strut portion after implantation and exposure to a magnetic field.

FIGS. 5A-5C show strut portions of a stent according to another embodiment. FIG. 5A shows a cross-sectional side view of the strut portion. FIG. 5B shows the strut portion after implantation and exposure to a magnetic field. FIG. 5C shows the strut portion with the therapeutic agent being released from the coating.

It is to be noted that certain features in these drawings have been exaggerated to more clearly show details thereof, such as, for example, the size of the polymer molecules relative to the thickness of the coatings.

### DETAILED DESCRIPTION

Medical devices of the present invention having a coating for the controlled release of therapeutic agents. Release of the therapeutic agent from the coating is facilitated or modulated by the application of an electromagnetic field (including electric and magnetic fields) to the medical device. As such, the release of the therapeutic agent can be triggered on-demand at a suitable time to increase the therapeutic effectiveness of the therapeutic agent and reduce unwanted adverse effects that the therapeutic agent may cause. For example, in the case of a vascular stent having a drug coating for the prevention of restenosis, release of the drug can be delayed until a time more suitable for the treatment of restenosis, which can occur weeks or months after the stent is implanted.

The source of the electromagnetic field may be located outside the patient's body (e.g., using an MRI apparatus) or within the patient's body (e.g., by using an intravascular lead connected to a source providing a varying electric field current to generate a magnetic field or by using an esophageal RF probe), and may be provided by various apparatuses, including a magnetic resonance imaging apparatus (MRI). The electromagnetic field may be static or time-varying (e.g., oscillating) so as to generate an electromagnetic wave (e.g., RF or microwave). In some cases, the electromagnetic field may be non-ionizing (e.g., low frequency RF) such that it does not cause damage to body tissue.

The coating comprises an ionic polymer (also known as an ion-conductive polymer), of which various types are known in the art, including sulfonated tetrafluoroethylene copolymers (e.g., Nafion® from DuPont) and ethylene-methacrylate copolymers (e.g., Surlyn® from DuPont). In some cases, the ionic polymer may also be electrically conductive. For example, the ionic polymer may have π-conjugated double-bonds along the backbone of the polymer to provide a conductive pathway along the polymer chain.

The medical device further comprises a therapeutic agent which is retained on the medical device by the coating. The therapeutic agent may be retained on the medical device by the coating in various ways, including being dispersed within the coating or being disposed under the coating. Application of an electromagnetic field to the medical device will cause a change in the ionic polymer and/or coating such that the therapeutic agent is released from the medical device. In some cases, the electromagnetic field can also induce an electric current through the coating, which may be created within the ionic polymer itself, through a metallic portion of the medical device that is in contact with the ionic polymer (e.g., the surface of a metal stent), or a combination of both. Electric currents passing through the ionic polymers may also play a role in the release of the therapeutic agent.

In certain embodiments, the ionic polymer undergoes an electrochemical change (e.g., oxidation or reduction) when exposed to an electromagnetic field. For example, an ionic polymer may have an electrostatic charge, with the electrostatic charge being reversed or neutralized upon exposure to an electromagnetic field. In some cases, the electrochemical change induced by the electromagnetic field is reversible when the electromagnetic field is removed or otherwise changed.

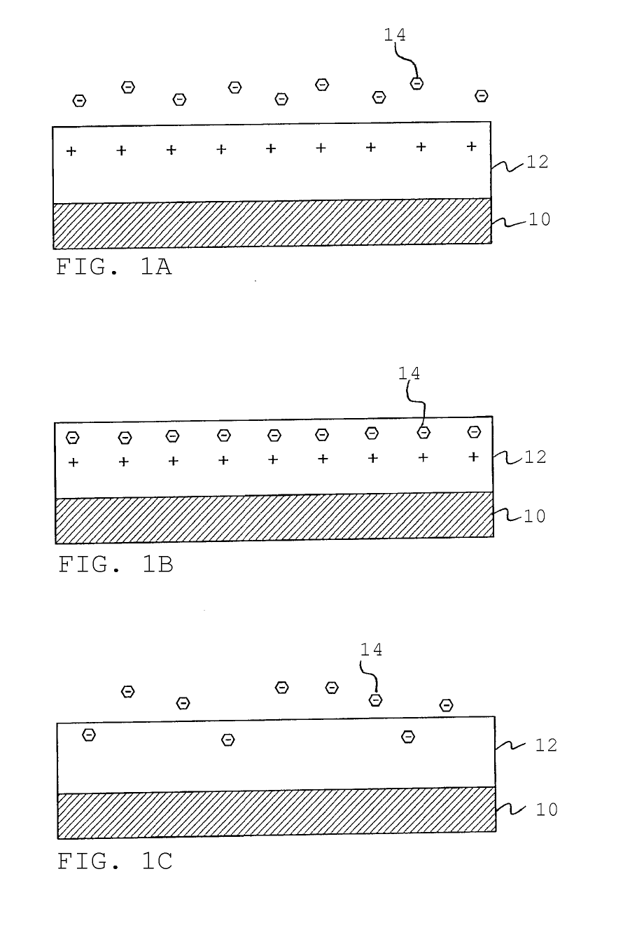

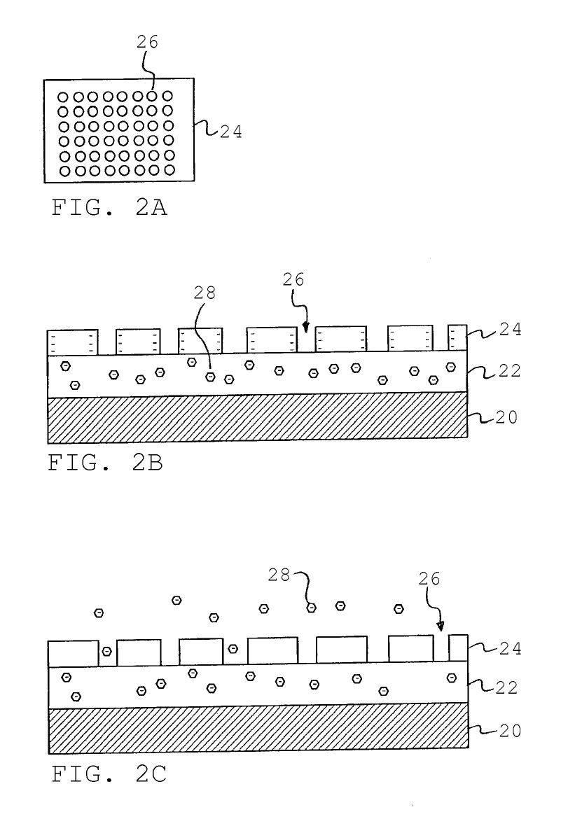

For example, in the embodiment shown in FIGS. 1A-1C, a strut portion 10 of a stent has a coating 12 comprising ionic polymers having reversible electrochemistry. As shown in FIG. 1A, the ionic polymers in coating 12 have a positive electrostatic charge. Referring to FIG. 1B, coating 12 is loaded with an anionic therapeutic agent 14 (acting as a counterion) which is driven into and held within the polymer matrix of coating 12 by electrostatic attraction to the positively-charged ionic polymers.

In operation, the stent is implanted into a blood vessel. When release of the therapeutic agent is desired, the stent is exposed to an electromagnetic field. As a result, as shown in FIG. 1C, the ionic polymers in coating 12 undergo an electrochemical change such that the electrostatic charges on the ionic polymers are neutralized. Freed from the electrostatic attraction to the ionic polymers, the anionic therapeutic agent 14 is released from coating 12.

In certain embodiments, the therapeutic agent is disposed under the coating and the coating acts as a selectively permeable membrane that controls the passage of the therapeutic agent through the coating. The therapeutic agent may be provided in various ways, including as the therapeutic agent formulation alone or with any structure that retains or holds the therapeutic agent. For example, the therapeutic agent may be dispersed within a polymer layer that is disposed under the coating or the therapeutic agent may be contained in pores, pits, cavities, or holes in the surface of the medical device.

For example, referring to the embodiment shown in FIGS. 2A-2C, a strut portion 20 of a stent has an inner layer 22 containing an anionic therapeutic agent 28. Disposed over inner layer 22 is a barrier coating 24 comprising ionic polymers, wherein barrier coating 24 serves as a membrane that selectively allows the passage of therapeutic agent 28 from inner layer 22. As seen in FIG. 2A (top view) and FIG. 2B (cross-sectional side view), barrier coating 24 has a plurality of micro- or nano-sized ion-conducting channels 26 which are capable of transporting anionic therapeutic agent 28. However, ion-conducting channels 26 are lined with negative electrostatic charges such that the transport of anionic therapeutic agent 28 is blocked.

In operation, the stent is implanted into a blood vessel. When release of the therapeutic agent is desired, the stent is exposed to an electromagnetic field. As a result, as shown in FIG. 2C, the electrochemistry of ionic polymers change such that the negative electrostatic charges lining ion-conducting channels 26 are neutralized. This allows the passage of anionic therapeutic agent 28 through ion-conducting channels 26.

In certain embodiments, the ionic polymer causes the coating to undergo structural changes when exposed to an electromagnetic field. Various types of structural changes in the coating are possible, including changes in its size (e.g., swelling) or shape. In some cases, stresses in the coating caused by these structural changes causes the release of the therapeutic agent.

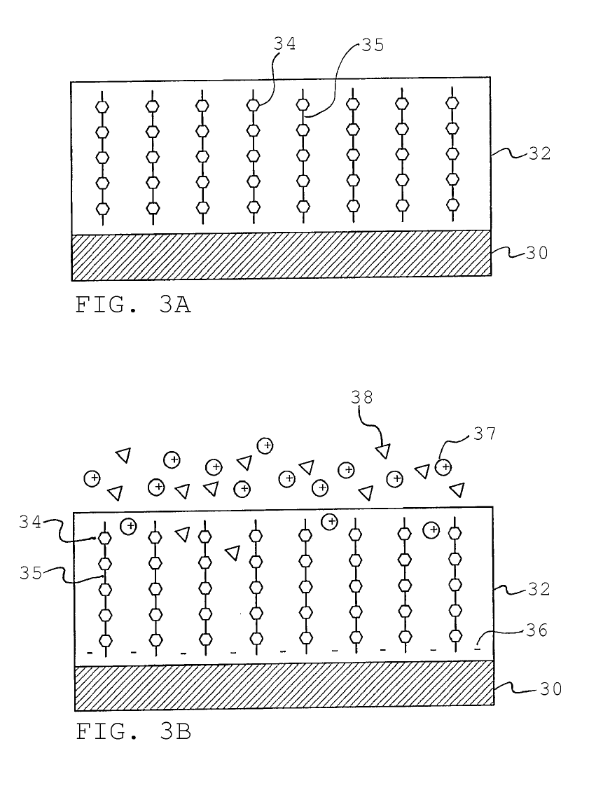

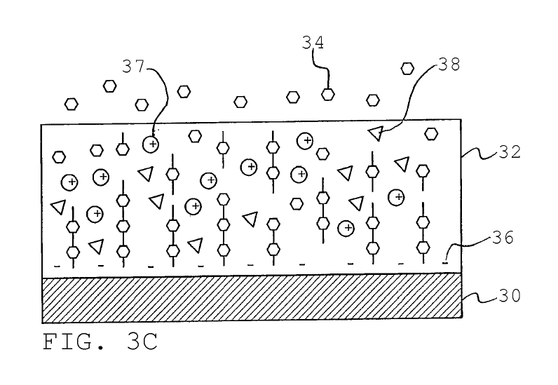

For example, in the embodiment shown in FIGS. 3A-3C, a strut portion 30 of a stent has a coating 32 comprising ionic polymers which undergo reversible electrochemical changes under an electromagnetic field. As shown in FIG. 3A, in the absence of an applied electromagnetic field, the ionic polymers in coating 32 have no electrostatic charge. Coating 32 is loaded with a therapeutic agent 34, which form lamellae sheets 35 within coating 32.

In operation, the stent is implanted into a blood vessel. When release of the therapeutic agent is desired, the stent is exposed to an electromagnetic field. As shown in FIG. 3B, under the electromagnetic field, the electrochemistry of the ionic polymers change such that the ionic polymers gain a negative electrostatic charge 36. Under the attraction of this negative electrostatic charge, cations 37 and water molecules 38 in the blood are drawn into coating 32. As shown in FIG. 3C, entry of these cations 37 and water molecules 38 causes coating 32 to swell, imposing stress upon coating layer 32 such that lamellae sheets 35 break apart with release of therapeutic agent 34.

In certain embodiments, the ionic polymer is sensitive to a magnetic field. As such, the application of a magnetic field to the medical device will cause the ionic polymers to become aligned or undergo motion under the magnetic field.

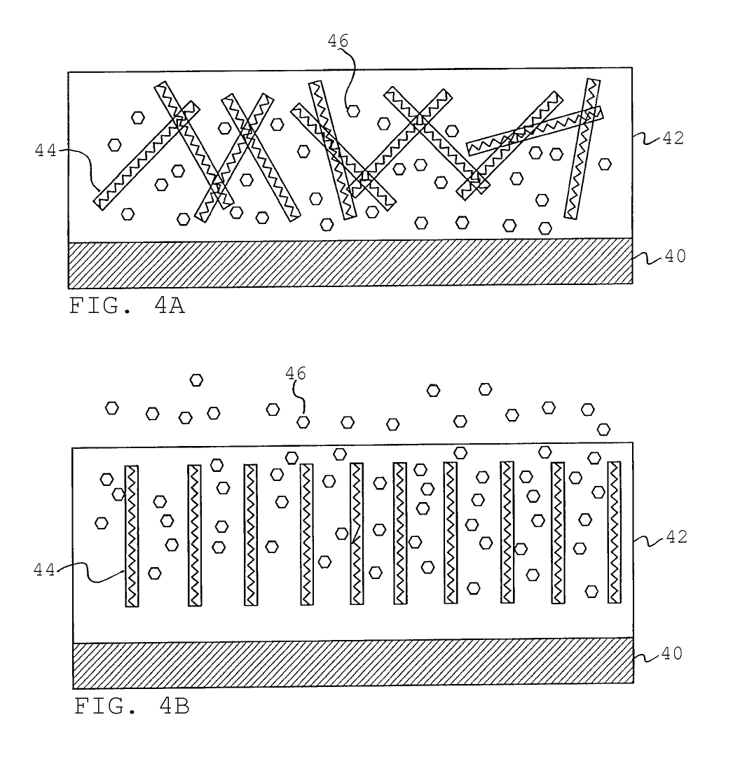

For example, in the embodiment shown in FIGS. 4A and 4B, a strut portion 40 of a stent has a coating 42 comprising magnetically-sensitive ionic polymers 44. As shown in FIG. 4A, magnetically-sensitive ionic polymers 44 are arranged in various orientations (which may be random). Therapeutic agent 46 is dispersed within coating 42 and trapped within the matrix of magnetically-sensitive polymers 44.

In operation, the stent is implanted into a blood vessel. When release of the therapeutic agent is desired, the stent is exposed to a magnetic field. As shown in FIG. 4B, under the magnetic field, magnetically-sensitive ionic polymers 44 in coating 42 become aligned with the magnetic field such that they are oriented in a uniform direction. This uniform orientation of magnetically-sensitive ionic polymers 44 creates passageways for therapeutic agent 46 to travel between magnetically-sensitive polymers 44 and be released from coating 42.

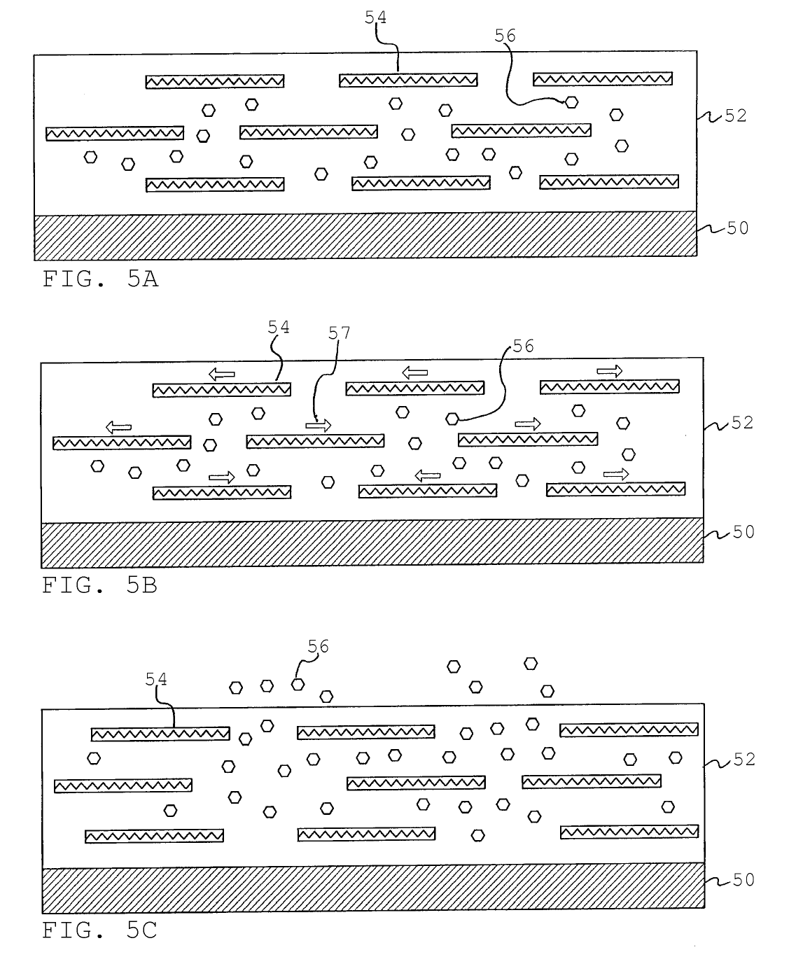

In another example, in the embodiment shown in FIGS. 5A-5C, a strut portion 50 of a stent has a coating 52 comprising magnetically-sensitive ionic polymers 54. As shown in FIG. 5A, therapeutic agent 56 is dispersed within coating 52 and trapped within the matrix of magnetically-sensitive ionic polymers 54.

In operation, the stent is implanted into a blood vessel. When release of the therapeutic agent is desired, the stent is exposed to an alternating magnetic field. As shown in FIG. 5B, under the magnetic field, magnetically-sensitive ionic polymers 54 in coating 52 move according to their individual polarity and orientation (in the direction of arrow 57). This movement of magnetically-sensitive ionic polymers 54 agitates therapeutic agent 56 so that it diffuses through the gaps between magnetically-sensitive ionic polymers 54 and becomes released from coating 52. An alternating magnetic field induces reciprocal (back-and-forth) motion of magnetically-sensitive ionic polymers 54, facilitating further release of therapeutic agent 56.

In a specific embodiment of the present invention, the medical device is a stent having a coating formed of Nafion® (a sulfonated tetrafluorethylene copolymer having ionic properties) and 8.8 wt % paclitaxel (a therapeutic agent). The biocompatibility of Nafion® has been evaluated, as reported in Turner et al., “Preliminary in vivo biocompatibility studies on perfluorosulphonic acid polymer membranes for biosensor applications,” Biomaterials, vol. 12, pp. 361-368 (1991). The coating is of sufficient thickness to provide a paclitaxel dosing of about 1 μg/mm² of stent surface area. The coating can be formed using a dimethyl acetamide/tetrahydrofuran solvent mixture containing 2 wt % solid (polymer plus drug) as the coating solution. The coating solution can be applied to the stent by spray coating or dip coating.

Non-limiting examples of medical devices that can be used with the present invention include stents, stent grafts, catheters, guide wires, neurovascular aneurysm coils, balloons, balloon catheters, filters (e.g., vena cava filters), vascular grafts, intraluminal paving systems, pacemakers, electrodes, leads, defibrillators, joint and bone implants, spinal implants, access ports, intra-aortic balloon pumps, heart valves, sutures, artificial hearts, neurological stimulators, cochlear implants, retinal implants, and other devices that can be used in connection with therapeutic coatings. Such medical devices are implanted or otherwise used in body structures, cavities, or lumens such as the vasculature, gastrointestinal tract, abdomen, peritoneum, airways, esophagus, trachea, colon, rectum, biliary tract, urinary tract, prostate, brain, spine, lung, liver, heart, skeletal muscle, kidney, bladder, intestines, stomach, pancreas, ovary, uterus, cartilage, eye, bone, joints, and the like.

The therapeutic agent used in the present invention may be any pharmaceutically acceptable agent, a biomolecule, a small molecule, or cells. Exemplary biomolecules include peptides, polypeptides and proteins; antibodies; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Exemplary small molecules include hormones, nucleotides, amino acids, sugars, and lipids and compounds have a molecular weight of less than 100 kD. Exemplary cells include stem cells, progenitor cells, endothelial cells, adult cardiomyocytes, and smooth muscle cells.

A reference to an element by the indefinite article “a” or “an” does not exclude the possibility that more than one of the element is present. Rather, the article “a” or “an” is intended to mean one or more (or at least one) unless the text expressly indicates otherwise. The terms “first,” “second,” and so on, when referring to an element, are not intended to suggest a location or ordering of the elements. Rather, the terms are used as labels to facilitate discussion and distinguish elements from one another.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

We claim:

1. A medical device comprising:
a coating comprising an ionic polymer; and
a therapeutic agent retained by the coating;
wherein the therapeutic agent is released from the medical device when the medical device is exposed to an electromagnetic field.

2. The medical device of claim 1, wherein the ionic polymer undergoes an electrochemical change when exposed to the electromagnetic field.

3. The medical device of claim 2, wherein the electrochemical change is reversible.

4. The medical device of claim 2, wherein the ionic polymer has an electrostatic charge, and wherein the electrostatic charge is neutralized or reversed upon exposure to the electromagnetic field.

5. The medical device of claim 4, wherein the therapeutic agent is dispersed within the coating, and wherein the therapeutic agent has an electrostatic charge opposite to that of the ionic polymer.

6. The medical device of claim 1, wherein the coating is disposed over the therapeutic agent, and wherein the coating serves as a selectively permeable membrane for the therapeutic agent.

7. The medical device of claim 6, wherein the coating becomes permeable to the therapeutic agent when the medical device is exposed the electromagnetic field.

8. The medical device of claim 6, wherein the ionic polymer undergoes an electrochemical change when exposed to the electromagnetic field.

9. The medical device of claim 6, wherein the coating has a plurality of ion-conducting channels, and wherein the therapeutic agent is transported through the ion-conducting channels when the medical device is exposed to the electromagnetic field.

10. The medical device of claim 6, wherein the therapeutic agent is anionic or cationic.

11. The medical device of claim 1, wherein the coating undergoes a structural change when the medical device is exposed to the electromagnetic field.

12. The medical device of claim 11, wherein the structural change is swelling of the coating.

13. The medical device of claim 12, wherein the therapeutic agent forms lamellae sheets within the coating, and wherein the lamellae sheets break apart and release the therapeutic agent when the coating swells.

14. The medical device of claim 1, wherein the ionic polymers are sensitive to a magnetic field, and wherein the electromagnetic field is a magnetic field.

15. The medical device of claim 14, wherein the ionic polymers are non-uniformly oriented, and wherein at least some of the ionic polymers, upon exposure to the magnetic field, become aligned with the magnetic field.

16. The medical device of claim 14, wherein the ionic polymers undergo motion when exposed to the magnetic field.

17. The medical device of claim 1, wherein the medical device is a vascular stent.

18. A method for delivering a therapeutic agent, comprising:
providing a medical device comprising:
(a) a coating comprising an ionic polymer; and
(b) a therapeutic agent retained by the coating;
positioning the medical device at a site in a patient's body; and
applying an electromagnetic field to the medical device, wherein the application of the electromagnetic field causes the release of the therapeutic agent from the medical device.

19. The method of claim 18, wherein the step of applying an electromagnetic field comprises applying a time-varying electromagnetic field to the medical device.

20. The method of claim 19, wherein the time-varying electromagnetic field is an oscillating electromagnetic field.

21. The method of claim 18, wherein the step of applying an electromagnetic field comprises applying a static electromagnetic field to the medical device.

22. The method of claim 18, wherein the source of the electromagnetic field is external to the patient's body.
